(19) United States
(12) Patent Application Publication
(10) Pub. No.: US 2017/0360360 A1
(43) Pub. Date: Dec. 21, 2017

(54) SLEEP MONITORING CAP

(71) Applicant: Yousef ALQURASHI, San Marcos, TX (US)

(72) Inventor: Yousef ALQURASHI, San Marcos, TX (US)

(21) Appl. No.: 15/183,068

(22) Filed: Jun. 15, 2016

(51) Int. Cl. A61B 5/00; A61B 5/0496; A61B 5/0478; A61B 5/11; A61B 5/0492

(52) U.S. Cl. CPC A61B 5/4809; A61B 5/6803; A61B 5/1103; A61B 5/0006; A61B 5/0492; A61B 5/0478; A61B 5/0496

(57) ABSTRACT

A sleep-monitoring cap includes interconnected electrodes embedded within a body of the sleep-monitoring cap. The interconnected electrodes are located at positions across a central transverse region, below and along a side of each eye, and on a rear mid-region of a person's head when wearing the sleep-monitoring cap. The sleep-monitoring cap includes a vibratory device embedded within the sleep-monitoring cap, wherein the vibratory device is connected to the interconnected electrodes. The sleep-monitoring cap includes processing circuitry embedded within the sleep-monitoring cap. The processing circuitry is configured to monitor, convert, process, and store brain wave activity retrieved by the interconnected electrodes from the person wearing the sleep-monitoring cap; determine whether the monitored brain wave activity includes low amplitude mixed-frequency waves and if so, activate the vibratory device; determine whether the monitored brain wave activity includes theta waves followed by vertex sharp waves and if so, activate the vibratory device.

### BACKGROUND

### Grant of Non-Exclusive Right

This application was prepared with financial support from the Saudi Arabian Cultural Mission, and in consideration therefore the present inventor(s) has granted The Kingdom of Saudi Arabia a non-exclusive right to practice the present invention.

### Description of the Related Art

A large number of people have difficulties with falling asleep and maintaining sleep. Many people may experience frequent awakenings or they do not use their sleep time efficiently. The effects of even small amounts of sleep loss accumulate over time, which can result in a “sleep debt,” which manifests itself in the form of increasing impairment of alertness, memory, and decision-making. Vigilance, memory, decision-making, and other neurocognitive processes are all impacted by poor sleep quality, sleep deprivation, and accumulating sleep debt with potentially detrimental consequences.

Many people do not realize they are not sleeping well but nonetheless, suffer the consequences of inefficient sleep. Other people attempt to overcome sleep-related problems by taking sleep-inducing or sleep-assisting drugs, such as stimulants or using relaxation techniques prior to sleeping. While temporary amelioration of the effects of sleep deprivation can be achieved using some of these techniques, an adequate amount of sleep that is commensurate with a person's accumulated sleep debt is indispensable for complete recuperation in the long run.

Many situations do not allow for a regular bout of nocturnal sleep. In such situations, brief naps, taken at various times throughout the day, have been advocated as an effective and natural means of countering fatigue and improving performance. Unfortunately, it is not easy to devise an optimal schedule for napping. In addition, the effects of a nap on dexterity and cognition depend, not only upon its duration, but also upon the sleep quality, the timing or period on the circadian cycle (i.e., the human's genetic preference to perform certain physiological functions only at certain times of the day or night) at which the nap occurred, and the depth of sleep from which the subject is awakened.

Sleep occurs in various stages, and each stage has its attendant purpose and advantages. Adequate balance among the sleep stages over long periods of time is important. For example, a persistent lack of Rapid Eye Movement (REM) sleep can result in a decline in performance, even if the total sleep time per day appears adequate. Therefore, a sleep paradigm that only prescribes durations and/or frequencies for sleeping will not necessarily result in a consistent and effective mitigation of performance deficits.

Sleep occupies approximately one-third of our lives. It has been established that sleep or the lack thereof is associated with heart disease, diabetes, immune deficiency, and memory deficit. Current practices for assessing sleep disorders can be time consuming and financially intensive. Sleep disorder research is usually conducted in a sleep laboratory managed by practitioners where sleep disorders, such as narcolepsy and sleep apnea, can be diagnosed. Home sleep testing kits are available. However, many sleep disorders cannot be detected by home sleep testing kits.

Drowsiness also negatively impacts a large number of people. Deaths due to falling asleep while driving in the United States are estimated at 5600 per year. In addition, drowsiness can negatively impact other areas of life, such as work activities.

The “background” description provided herein is for the purpose of generally presenting the context of the disclosure. Work of the presently named inventor, to the extent it is described in this background section, as well as aspects of the description which may not otherwise qualify as prior art at the time of filing, are neither expressly nor impliedly admitted as prior art against the present disclosure.

### SUMMARY

In one embodiment, a sleep-monitoring cap includes a plurality of interconnected electrodes embedded within a body of the sleep-monitoring cap. The plurality of interconnected electrodes is located at positions across a central transverse region, below and along a side of each eye, and on a rear mid-region of a person's head when the person is wearing the sleep-monitoring cap. The sleep-monitoring cap also includes a vibratory device embedded within the body of the sleep-monitoring cap, wherein the vibratory device is connected to the plurality of interconnected electrodes. The sleep-monitoring cap also includes first processing circuitry embedded within the body of the sleep-monitoring cap. The first processing circuitry is configured to monitor, convert, process, and store a first set of brain wave activity retrieved by the plurality of interconnected electrodes from the person wearing the sleep-monitoring cap; determine whether a sleep state exists from the monitored first set of brain wave activity; when the sleep state exists, determine whether a first sleep stage is a REM sleep stage; when the first sleep stage is a REM sleep stage, record the sleep state as a sleep onset REM period; and activate the vibratory device after a pre-determined time period when the sleep onset REM period has been recorded. The sleep-monitoring cap also includes second processing circuitry embedded within the body of the sleep-monitoring cap. The second processing circuitry is configured to monitor, convert, process, and store a second set of brain wave activity retrieved by the plurality of interconnected electrodes from the person wearing the sleep-monitoring cap; determine whether the monitored second set of brain wave activity includes low amplitude mixed-frequency waves; when the monitored second set of brain wave activity includes the low amplitude mixed-frequency waves, activate the vibratory device; determine whether the monitored second set of brain wave activity includes theta waves followed by vertex sharp waves; and when the monitored second set of brain wave activity includes the theta waves followed by the vertex sharp waves, activate the vibratory device.

In one embodiment, a sleep-monitoring cap includes a plurality of interconnected electrodes embedded within a body of the sleep-monitoring cap. The plurality of interconnected electrodes is located at positions across a central transverse region, below and along a side of each eye, and on a rear mid-region of a person's head when the person is wearing the sleep-monitoring cap. The sleep-monitoring cap also includes a vibratory device embedded within the body of the sleep-monitoring cap, wherein the vibratory device is connected to the plurality of interconnected electrodes. The sleep-monitoring cap also includes processing circuitry embedded within the body of the sleep-monitoring cap. The processing circuitry is configured to monitor, convert, process, and store brain wave activity retrieved by the plurality of interconnected electrodes from the person wearing the sleep-monitoring cap; determine whether a sleep state exists from the monitored brain wave activity; when the sleep state exists, determine whether a first sleep stage is a REM sleep stage; when the first sleep stage is a REM sleep stage, record the sleep state as a sleep onset REM period; and activate the vibratory device after a pre-determined time period when the sleep onset REM period has been recorded.

In one embodiment, a sleep-monitoring cap includes a plurality of interconnected electrodes embedded within a body of the sleep-monitoring cap. The plurality of interconnected electrodes is located at positions across a central transverse region, below and along a side of each eye, and on a rear mid-region of a person's head when the person is wearing the sleep-monitoring cap. The sleep-monitoring cap also includes a vibratory device embedded within the body of the sleep-monitoring cap, wherein the vibratory device is connected to the plurality of interconnected electrodes. The sleep-monitoring cap also includes processing circuitry embedded within the body of the sleep-monitoring cap. The processing circuitry is configured to monitor, convert, process, and store brain wave activity retrieved by the plurality of interconnected electrodes from the person wearing the sleep-monitoring cap; determine whether the monitored brain wave activity includes low amplitude mixed-frequency waves; when the monitored brain wave activity includes the low amplitude mixed-frequency waves, activate the vibratory device; determine whether the monitored brain wave activity includes theta waves followed by vertex sharp waves; and when the monitored brain wave activity includes the theta waves followed by the vertex sharp waves, activate the vibratory device.

The foregoing paragraphs have been provided by way of general introduction, and are not intended to limit the scope of the following claims. The described embodiments will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 illustrates an overhead view of electrode positioning of a sleep-monitoring cap on a person's head according to one embodiment;

FIG. 2 illustrates a back view of electrode positioning of a sleep-monitoring cap on a person's head according to one embodiment;

FIG. 3 illustrates a side view of electrode positioning and a sleeping cap positioning on a person's head according to one embodiment;

FIG. 4 illustrates a front view of electrode positioning and a sleeping cap positioning on a person's head according to one embodiment;

FIG. 5 is an exemplary algorithm for monitoring and determining brain wave activity according to one embodiment;

FIG. 6 is an exemplary algorithm for monitoring drowsiness according to one embodiment;

FIG. 7 illustrates an exemplary sleep-monitoring cap with an embedded transistor according to one embodiment;

FIG. 8 illustrates a hardware description of an exemplary computing device according to one embodiment;

FIG. 9 is a schematic diagram of an exemplary data processing system according to one embodiment; and

FIG. 10 is a schematic diagram of an exemplary central processing unit (CPU) according to one embodiment.

### DETAILED DESCRIPTION

The science of sleep distinguishes five stages of sleep, including wakefulness as a pre-sleep stage. There are three stages of non-rapid eye movement (NREM), which are stage 1, stage 2, and stage 3. There is also a rapid eye movement (REM) stage. The different stages of sleep can be identified using various techniques to monitor brain wave patterns, such as using an electroencephalogram (EEG) technique, monitoring eye movements using an electro-oculogram (EOG) technique, and monitoring the movements of the chin using electromyogram (EMG) techniques.

A rested wakeful stage is characterized by low amplitude alpha waves (8-12 Hz) present in an EEG of a person whose brain waves are being monitored. Alpha waves are brain waves typically exhibited while a person is in a wakeful and relaxed state with the person's eyes being closed. The alpha waves typically decrease in amplitude while the person's eyes are opening or the subject is in a drowsy or sleeping state.

NREM Stage 1 is characterized by irregular theta waves of low amplitude present in the EEG of a person. Slow rolling eye movements are also present in an EOG of the subject.

NREM Stage 2 is characterized by high frequency (12-16 Hz) bursts of brain activity called sleep spindles riding on top of slower brain waves of higher amplitude. During NREM Stage 2, a gradual decline in heart rate, respiration, and core body temperature occurs as the body prepares to enter deep sleep.

NREM Stage 3 is characterized by delta waves (1-3 Hz) of large amplitude that dominate for more than 20% of the time.

REM sleep presents a marked drop in muscle tone and bursts of rapid eye movements that can be seen in the EOG. The EEG in REM is not specific and resembles that of wakefulness or NREM Stage 1 sleep. Other physiological signals (e.g. breathing, heart rate) during REM sleep also exhibit a pattern similar to that occurring in an awakened individual.

Sleep stages come in cycles that repeat on the average of four to six times a night, with each cycle lasting approximately ninety to one hundred twenty minutes. The order of the stages of a sleep cycle and the length of the sleep stages may vary from person to person and from sleep cycle to sleep cycle. For example, NREM Stage 3 may be more prevalent during sleep cycles that occur early in the night, while NREM Stage 2 and REM sleep stages may be more prevalent in sleep cycles that occur later in the night. The sequence and/or length of sleep stages during an overnight sleep or nap is sometimes interrupted with brief periods of wakefulness. This makes up a person's sleep architecture.

A balanced sleep architecture is important, especially during a nap or shortened period of sleep because the various stages of sleep contribute differently to recuperation. A sleep period composed only of light sleep (NREM Stage 1) does not improve performance, whereas even a few minutes of solid sleep (NREM Stage 2) can boost alertness, attention, and motor performance. Deep sleep (NREM Stage 3) is desirable because it reduces stress and improves skill acquisition. However, interruptions during NREM Stage 3 sleep can lead to decrements in performance. A persistent lack of REM sleep can result in a decline in performance, even if the total sleep time per day appears to be adequate.

One of the common sleep disorders is narcolepsy, which is a result of abnormal REM sleep. Narcoleptics generally experience the REM stage of sleep within five minutes of falling asleep, while a non-narcoleptic does not experience REM in the first hour or so of a sleep cycle until after a period of slow-wave sleep. Narcoleptics commonly experience frequent excessive daytime sleepiness, comparable to how non-narcoleptics feel after 24 to 48 hours of sleep deprivation. The disturbed nocturnal sleep is often confused with insomnia. Narcoleptics can also experience cataplexy, which is a sudden and transient episode of muscle weakness or total loss of muscle tone accompanied by full conscious awareness, typically triggered by emotions such as laughing, crying, tenor, etc.

Embodiments herein describe methods and systems for monitoring sleep and sleep disorders. An embodiment describes a method of monitoring REM sleep patterns, which includes applying electrodes to a person's head, wherein the electrodes are configured to measure a plurality of sleep stages of the person during a sleep state. The method also includes determining how long after falling asleep the person enters REM sleep. When the person enters REM sleep as a first sleep stage, the method includes waking the person up approximately 20 minutes later using a vibrator, which is electronically attached to the electrodes. If the sleep did not occur, the test will end after approximately 20 minutes. The method also includes receiving data results of the plurality of sleep stages, via a memory device.

Another embodiment describes an electrode cap, which is configured to determine narcolepsy in a person. The cap is configured to fit over a person's head during sleep, and includes a plurality of electrodes in the cap configured to measure different stages of sleep of the person. The cap also includes a vibrator, which is configured to wake the person up after approximately 20 minutes when REM sleep is entered as a first sleep stage. The cap also includes a memory device to record the different stages of sleep activity of the person.

Another embodiment describes an electrode cap, which is configured to measure a drowsiness stale. The cap is configured to fit over a person's head, and includes a plurality of electrodes in the cap configured to measure brain wave activity of a person wearing the cap. The cap also includes a vibrator, which is configured to vibrate when slow eye movement followed by theta EEG waves are measured by the cap. The vibrator is also configured to vibrate when the cap measures theta waves followed by vertex sharp waves.

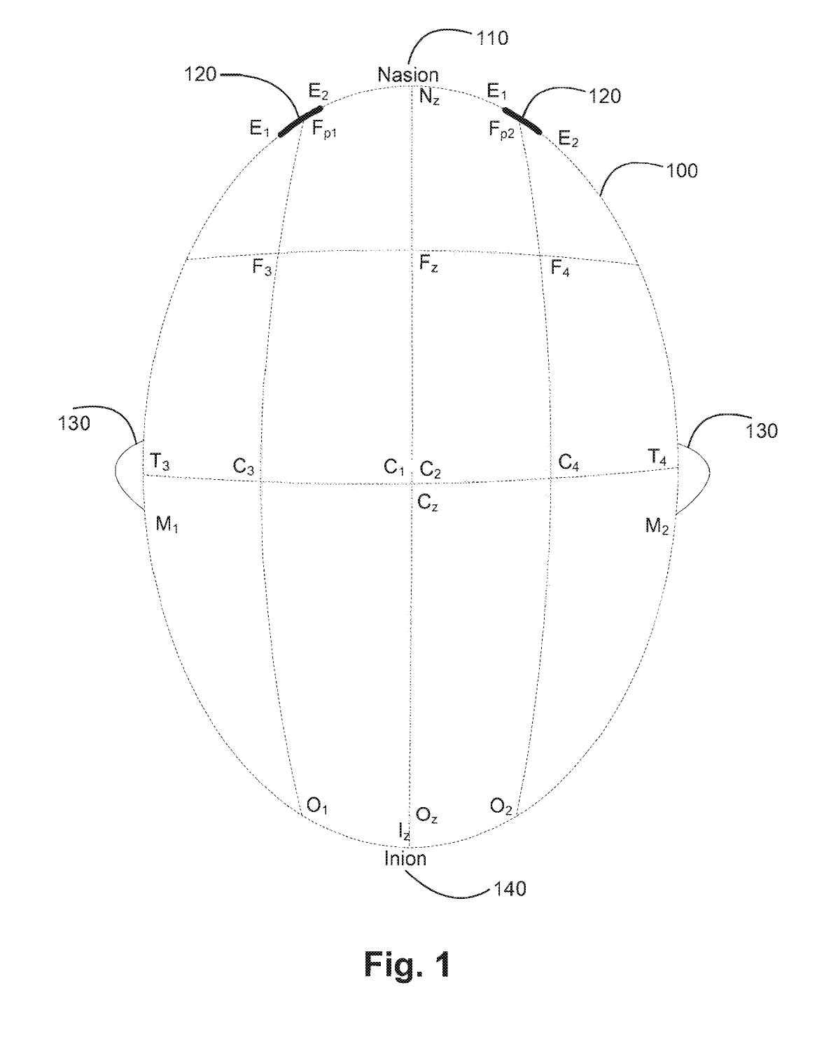

FIG. 1 illustrates an overhead view of electrodes positioned on a head 100 of a person. A front end 110 of head 100 includes two eyes 120, and each side of head 100 includes an ear 130. A back end 140 of head 100 is also illustrated. Head 100 illustrates the placement of electrodes for purposes of measuring sleep states of the person via an electro-encephalogram (EEG). The illustrated electrode notations use the standard naming and positioning scheme in the 10-20 international system for EEG applications. However, other naming conventions are contemplated by embodiments described herein. Even though several other electrodes have been established for use in an EEG for various purposes, embodiments described herein for measuring different sleep stages utilize the electrodes illustrated in FIG. 1. All electrodes at the notated positions in FIG. 1 are interconnected, such that electrical signals from the electrodes can be transmitted and recorded, via interconnecting electrical transmission times and a memory transistor chip, respectively.

An EEG measures brain waves by applying multiple electrodes to various positions on the head, as illustrated in FIG. 1. An EEG amplifier measures voltage differences between points on the scalp against reference points, M1 or M2, depending on the place of the active electrode, thereby creating a channel between two connected electrodes. EEG electrodes are small metal plates that are attached to the scalp. This can be accomplished by using a conducting electrode gel. In other embodiments, an elasticized cap fitted to a person's head can be used to hold the electrodes next to the scalp. The electrodes can be made from various materials, such as tin and silver/silver-chloride electrodes. Gold and platinum electrodes can also be used, as well as other conducting materials.

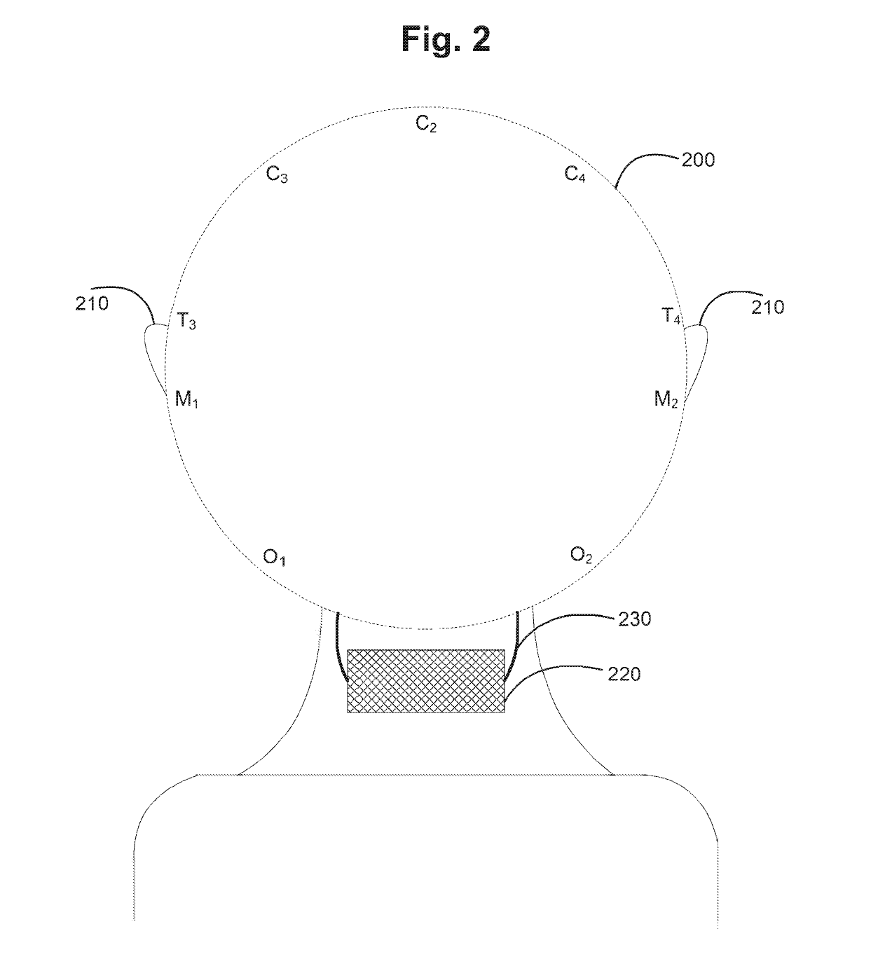

FIG. 2 illustrates a back view of electrodes positioned on a person's head 200. FIG. 2 illustrates electrodes from the crown of the head 200 towards the lower backside of the head 200. Ears 210 are illustrated on either side of the head 200 to better illustrate positioning of the electrodes. The illustrated electrodes are interconnected, along with electrodes on the front side of the head, as illustrated in FIG. 1. FIG. 2 also illustrates a vibrator 220, which is connected to the circuitry of the interconnected electrodes, via vibrator connectors 230.

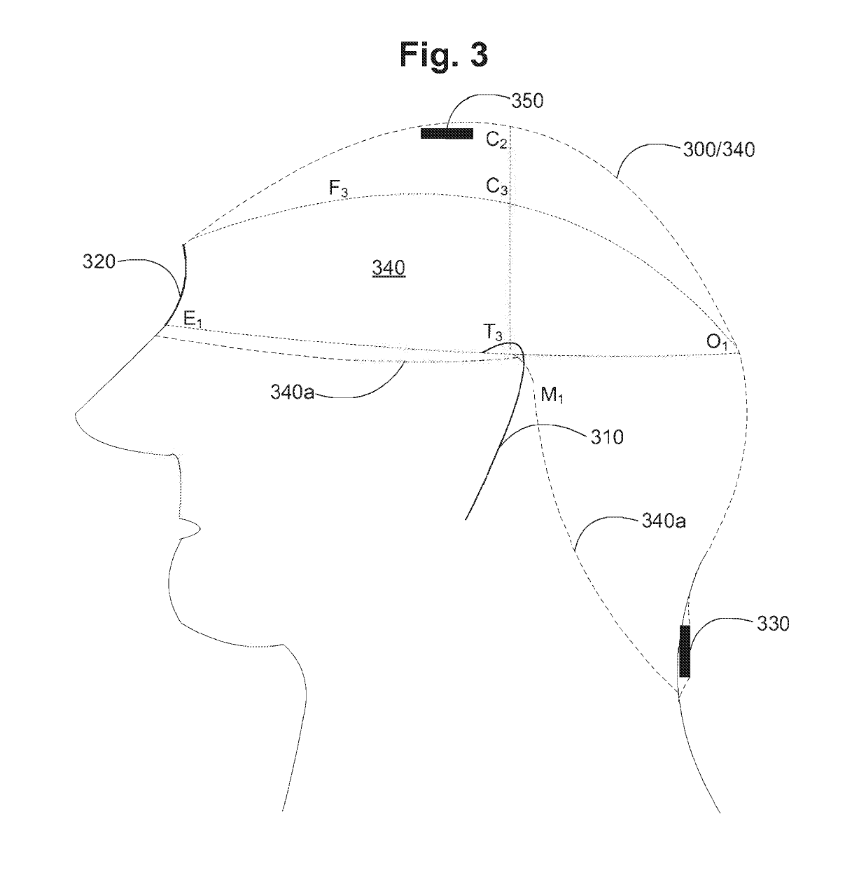

FIG. 3 illustrates a side view of electrodes positioned on a person's head 300 as viewed from a side perspective. An ear 310 and an eye 320 are also illustrated. FIG. 3 also illustrates a vibrator 330 on the lower back side of the head 300, which is connected by circuitry to the interconnected electrodes. Vibrator 330 is also held flush against the back side of the person's head 300. Vibrator 330 could be positioned just below the hairline of the person's head 300, although this is not a requirement.

FIG. 3 also illustrates a cap 340 that contains the electrodes illustrated in FIG. 1. The electrodes are embedded in the material of the cap 340, such that each electrode maintains its intended position flush against the person's head 300 with respect to all surrounding electrodes. The vibrator 330 is contained within the lower back region of the cap 340, such that the vibrator 330 is held against the person's head below or near to the hairline of the person's head 300. Edges 340a of the cap 340 are illustrated to run below the eye 320, behind the ear 310, and down and around the neck. Eye holes can be provided within the cap 340 to allow the person to see while wearing the cap 340.

Cap 340 also contains a processor 350 for recording and storing data from the electrical signals of the electrodes and for processing and analyzing the signals from the electrodes. The processor 350 could be located on the top side of the cap 340 as illustrated in FIG. 3 or another position, such that it would not provide any discomfort to the person from lying directly on the processor 350 while sleeping. A rechargeable battery-operated power source could also be included in the cap 340.

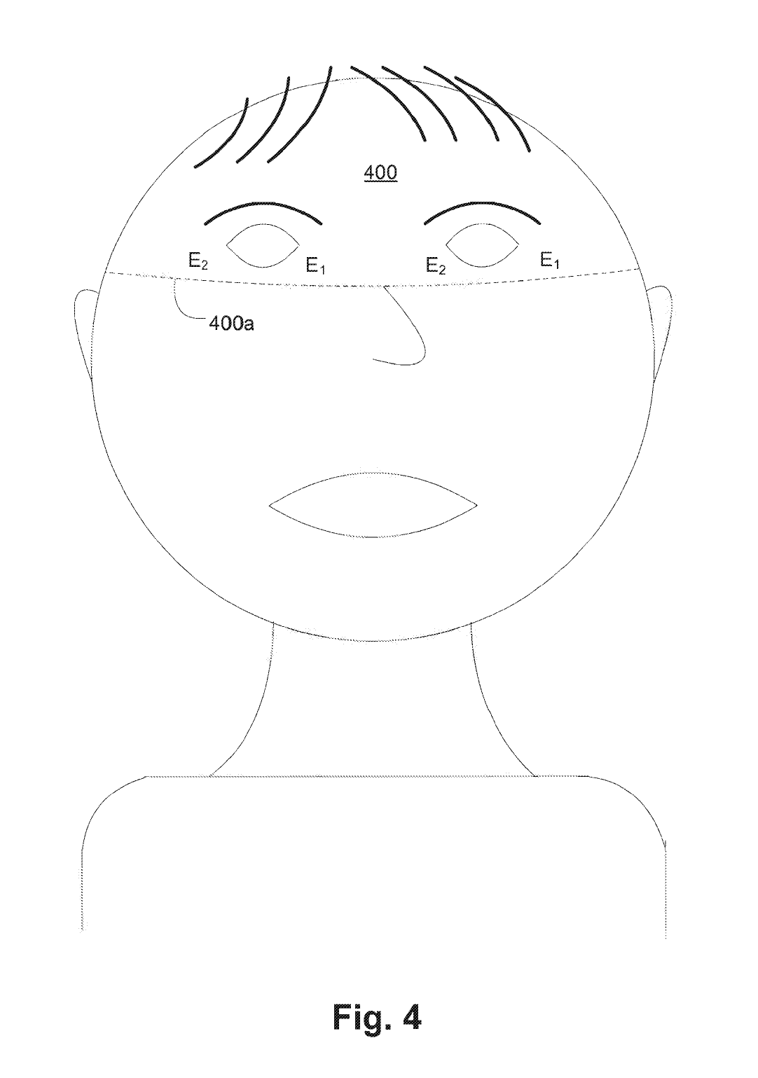

FIG. 4 illustrates a front view of electrodes and a cap 400 positioned on a person's head, wherein an edge 400a of the cap 400 runs below the eyes and above the nose and ears. Cap 400 properly places associated eye electrodes near the lower edges of the eyes while the person sleeps. Eye holes in the cap 400 would allow the person to see while wearing the cap 400. However, cap 400 could also be configured without eye holes to aid the person in sleeping by providing a dark environment.

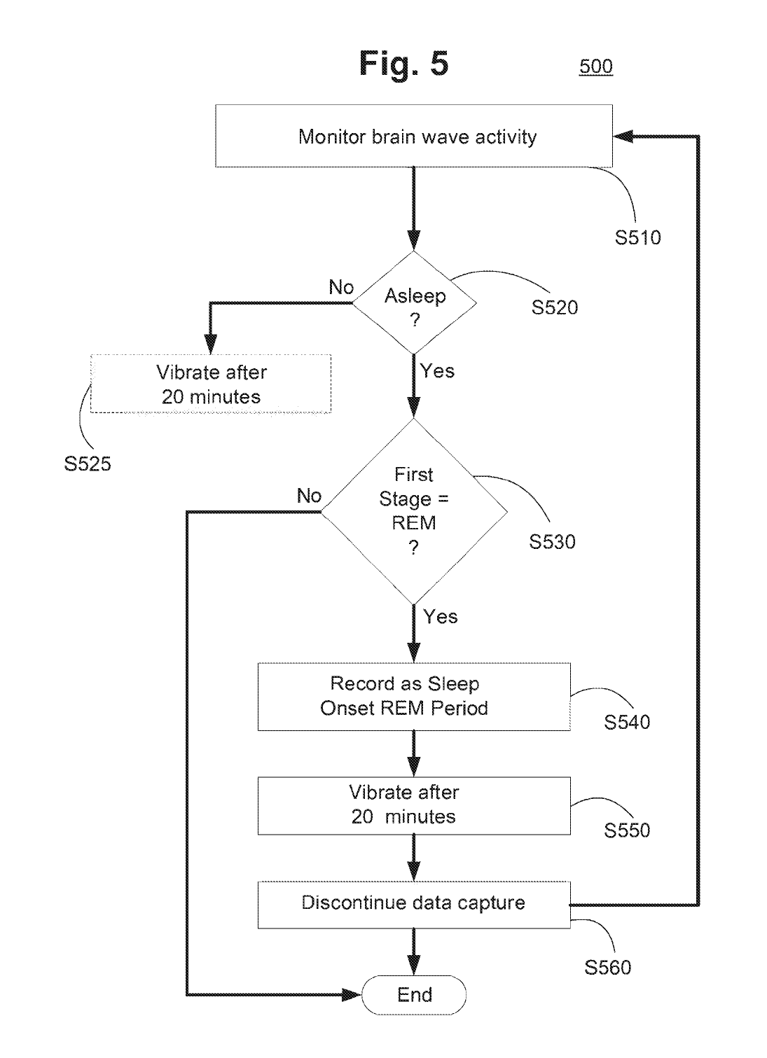

FIG. 5 is an exemplary algorithm 500 for monitoring and determining brain wave activity of a person, using at least in part, embodiments described above for a sleep-monitoring cap. In an embodiment, the algorithm 500 monitors and determines narcolepsy in a person wearing the sleep-monitoring cap. Exemplary algorithm 500 is implemented, via a sleep-monitoring cap configured with circuitry to perform the following algorithmic steps.

In step S510, a person's brain-wave activity is monitored, via a sleep-monitoring cap, such as the cap described above with reference to FIGS. 1-4. In step S520, it is determined whether the person is asleep. If the person is still not asleep after approximately 20 minutes, a vibrator contained within the cap vibrates in optional step S525, using the vibrator described above with reference to FIGS. 2-3. In an embodiment, it may be desirable for the person to rise up and move about before resuming the sleep monitoring.

If the person is asleep (a “yes” decision in step S520), the process continues to step S530. In step S530, it is determined whether the first stage of sleep entered from a waking state is REM. If the first stage is not REM (a “no” decision in step S530), the process ends. If the first stage is REM (a “yes” decision in step S530), the process continues to step S540 where a Sleep Onset REM Period (SOREMP) is recorded.

The process continues to step S550, where a vibration occurs approximately 20 minutes after SOREMP is detected, using the vibratory device embedded in the cap. In an embodiment, a wake-up period approximately 20 minutes after entering SOREMP is conducted in narcolepsy testing. However, other waiting periods after entering SOREMP are contemplated by embodiments described herein.

In step S560, data capture is discontinued after the vibratory device is activated, i.e. the person has been awakened. Brain activity continues to be monitored as long as the sleep-monitoring cap is activated in order to capture any additional SOREMP activity during a single sleep session. Therefore, the process begins again at step S510.

The vibrator, such as vibrator 220 or 330 can be programmed, via cap processor 350 with various options. For example, the vibrator can continue to vibrate until motion is detected or until a waking state is monitored by processor 350. If the person does not move or a waking state is not detected by processor 350 after a pre-determined amount of time, a different type of vibration and/or a different intensity of vibration could commence to awaken the person.

Measuring EEG waves is an objective way to distinguish between a sleep state and a wakefulness state. The different sleep stages, as well as a wakefulness state are associated with specific EEG wave activity.

A wakefulness state is associated with beta activity in the range of 12-40 Hertz.

Stage 1 sleep is associated with slow eye movement (SEM) and theta waves in the range of 4-7 Hertz. Stage 1 sleep can also be associated with vertex sharp waves coming from the central lobe (illustrated as Cz in FIG. 1) with an amplitude of 50-150 microVolts. Stage 1 sleep is also associated with alpha attenuation, where alpha waves become slower and less prominent. This stage is sometimes called a drowsiness stage or a transition stage from wakefulness to sleep. Stage 1 usually lasts for 2-5 minutes before stage 2 begins.

Stage 2 sleep is associated with K complex and sleep spindle. This stage is the most prominent stage in a normal sleeper.

Stage 3 sleep is associated with delta activity. Delta activity is very slow and is usually in the range of 0-4 Hertz. This stage is associated with memory consolidation.

Stage REM sleep is associated with high frequency low amplitude waves in which the brain becomes very active. REM stage sleep frequently exhibits alpha activity. REM stage sleep can he distinguished by rapid eye movement and a drop in muscle activity.

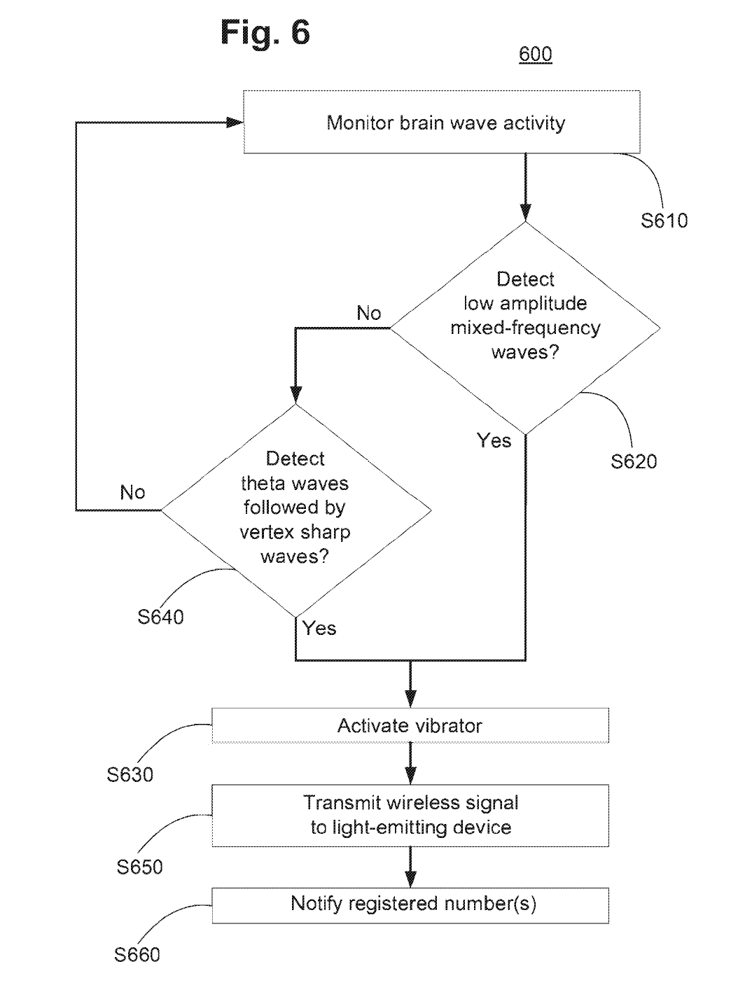

FIG. 6 is an exemplary algorithm 600 for monitoring the drowsiness of a person, using at least in part, embodiments described above for a sleep-monitoring cap. In an embodiment, the algorithm 600 monitors and determines a state of drowsiness by monitoring Stage 1 sleep. Exemplary algorithm 600 is implemented, via a sleep-monitoring cap configured with circuitry to perform the following algorithmic steps.

In step S610, brain wave activity is monitored by a sleep-monitoring cap, such as the sleep-monitoring cap described with reference to FIGS. 1-4. In step S620, it is determined whether low amplitude mixed-frequency waves are detected by the sleep-monitoring cap. This can be determined by detecting SEM followed by theta EEG waves in the frequency range of 4-7 Hertz. If low amplitude mixed-frequency waves are detected (a “yes” decision in step S620), a vibratory device, such as vibrator 220 or 330 is activated in step S630 to awaken the person wearing the sleep-monitoring cap.

If low amplitude mixed-frequency waves are not detected (a “no” decision in step S620), it is determined whether theta waves followed by vertex sharp waves are detected in step S640. If theta waves followed by vertex sharp waves are detected (a “yes” decision in step S640), a vibratory device, such as vibrator 220 or 330 is activated in step S630 to awaken the person wearing the sleep-monitoring cap. If theta waves followed by vertex sharp waves are not detected (a “no” decision in step S640), the process resumes at step S610 where brain wave activity continues to be monitored as long as the sleep-monitoring cap is activated.

In an additional embodiment, when the vibrator has been activated in step S630, a wireless signal can be transmitted, via an electronic transmitter embedded in the sleep-monitoring cap, to a light-emitting device in step S650. The light-emitting device can be worn on the sleep-monitoring cap, such as the back side or the front side. When the light-emitting device is activated, it would inform individuals surrounding the person wearing the sleep-monitoring cap that the person is entering into a drowsy state. This would inform surrounding individuals of an impending problem, and allow the surrounding individuals to possibly offer assistance. The light-emitting device can be a steady light or a flashing light.

The light-emitting device can also be affixed to a rear side of a vehicle in which the person is driving. When the light-emitting device becomes activated, it would inform other surrounding drivers of a possible impending problem. This would allow other drivers to provide more distance between the vehicle with the light-emitting device and other drivers' vehicles. The light-emitting device can be a single device or interconnected multiple devices. The light-emitting device(s) can be similar to emergency flashers of a vehicle.

In an additional embodiment, one or more registered numbers can be contacted in step S660, subsequent to steps S630 and S650. One of the registered numbers could be a mobile phone number of the person wearing the sleep-monitoring cap. In essence, this would provide a “wake-up call” to the person wearing the sleep-monitoring cap when a drowsy state has been detected. In addition, other landline or mobile numbers could be registered to inform other individuals of the person's drowsy state, such as a spouse, a supervisor, a medical practitioner or medical personnel, or a local law enforcement agency.

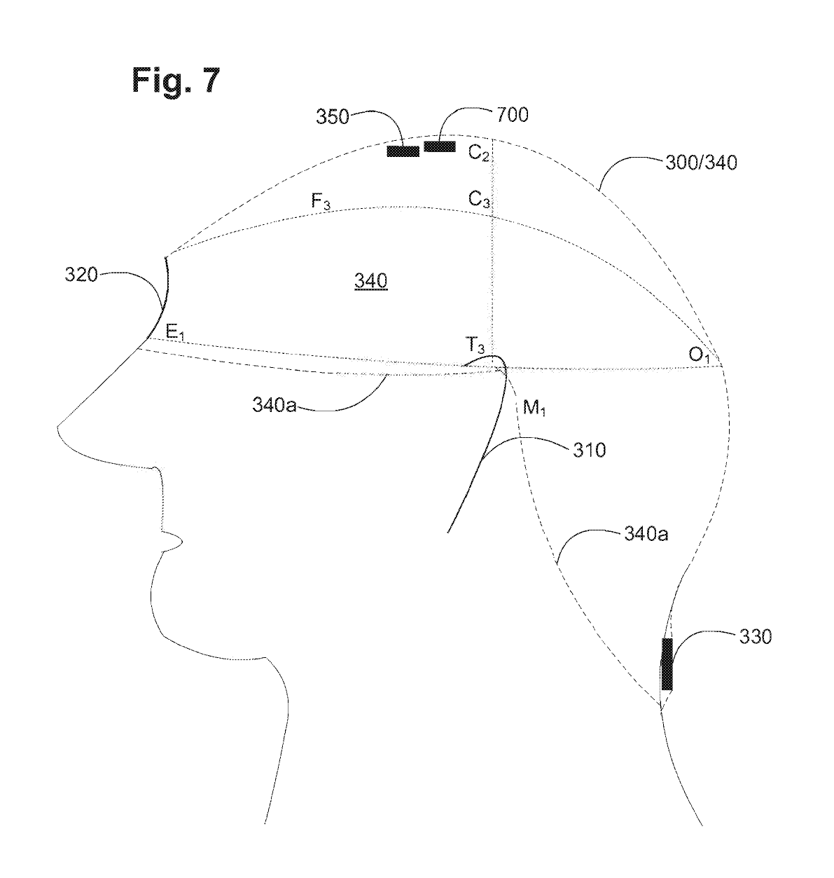

FIG. 7 illustrates an exemplary sleep-monitoring cap in which a transmitter 700 is embedded in the sleep-monitoring cap. The transmitter 700 could be located adjacent to the processor 350 or included within the same electronic device as the processor 350. In another embodiment, the transmitter 700 could be located in other areas of the sleep-monitoring cap, such as near or adjacent to the vibrator 330. The transmitter 700 is used in conjunction with a receiving light-emitting device located on the sleep-monitoring cap or located at another location, such as the rear side of a vehicle.

The structure of the sleep-monitoring cap in which the electrodes, the processor 350, the vibrator 330, and the transmitter 700 are embedded includes a material or fabric configured to firmly hold the electrodes close to the scalp of the person wearing the sleep-monitoring cap. The material or fabric would be stretchable, so as to provide a snug fit and maintain the position of each electrode close to the scalp without moving. In addition, a stretchable material or fabric would allow an adequate fit for multiple sizes and shapes of heads. The material or fabric could include nylon or polyester, designed to stretch when a force is applied to it, and return to its original shape and size when the force is removed. Embodiments also include a mesh material in which multiple open spaces or pores exist within the sleep-monitoring cap.

One or more straps affixed to the sleep-monitoring cap could also be configured to help maintain the snug fit. In an embodiment, a pair of straps could extend from the frontal lobe area of the head and snap or tie under the chin. In another embodiment, a pair of straps could extend from a rear area of the head behind the ears and snap or tie under the chin.

The sleep-monitoring cap can also be designed to be thin and fit under a hat. This would be advantageous for monitoring drowsiness when the person is amongst other individuals and does not wish for the sleep-monitoring cap to be viewed by others.

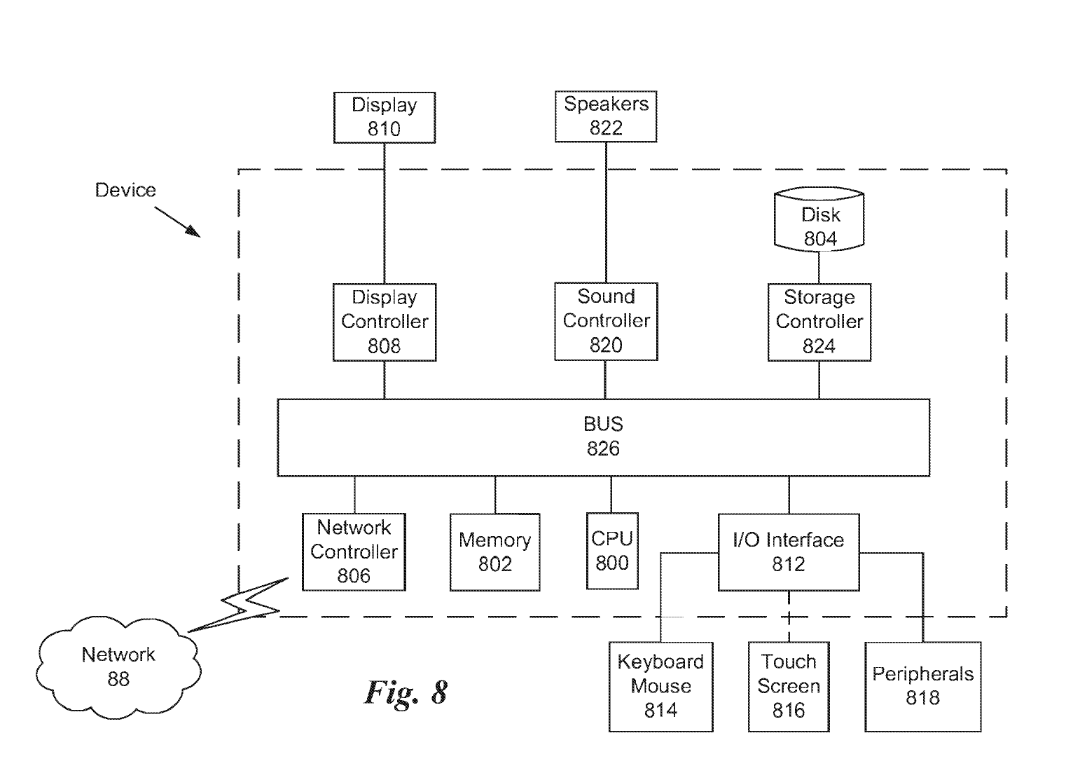

A hardware description is given with reference to FIG. 8 of a computing device, such as processor 350 and transmitter 700, which is used in conjunction with associated circuitry for embodiments described herein. The circuitry represents hardware and software components whereby the “configured by circuitry,” “configured by programming and circuitry,” and/or “configured to” elements of the disclosures noted herein are programmed. The programming in hardware and software constitutes algorithmic instructions to execute the various functions and acts noted and described herein. The computing device described herein can include one or more types of wireless and/or portable computing devices. The computing device described herein can also include physically separated devices that operate within a network.

In FIG. 8, the computing device includes a CPU 800 which performs the processes described above. The process data and instructions may be stored in memory 802. These processes and instructions may also be stored on a storage medium disk 804 such as a hard disc drive (HDD) or portable storage medium, or may be stored remotely. Further, the claimed embodiments are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the computing device communicates.

Further, the claimed embodiments may be provided as a utility application, background daemon, or component of an operating system, or combination thereof, executing in conjunction with CPU 800 and an operating system such as Microsoft Windows 7, UNIX, Solaris, LINUX, Apple MAC-OS and other systems known to those skilled in the art.

CPU 800 may be a Xenon or Core processor from Intel of America or an Opteron processor from AMD of America, or may be other processor types that would be recognized by one of ordinary skill in the art. Alternatively, the CPU 800 may be implemented on a Field Programmable Grid-Array (FPGA), Application-Specific Integrated Circuit (ASIC), Programmable Logic Device (PLD), or using discrete logic circuits, as one of ordinary skill in the art would recognize. Further, CPU 800 may be implemented as multiple processors cooperatively working in parallel to perform the instructions of the inventive processes described above.

The computing device in FIG. 8 also includes a network controller 806, such as an Intel Ethernet PRO network interface card from Intel Corporation of America, for interfacing with network 88. As can be appreciated, the network 88 can be a public network, such as the Internet, or a private network such as an LAN or WAN network, or any combination thereof and can also include PSTN or ISDN sub-networks. The network 88 can also be wired, such as an Ethernet network, or can be wireless such as a cellular network including EDGE, 3G and 4G wireless cellular systems. The wireless network can also be WiFi, Bluetooth, or any other wireless form of communication that is known.

The computing device further includes a display controller 808, such as a NVIDIA GeForce GTX or Quadro graphics adaptor from NVIDIA Corporation of America for interfacing with display 810, such as a Hewlett Packard HPL2445w LCD monitor. A general purpose I/O interface 812 interfaces with a keyboard and/or mouse 814 as well as a touch screen panel 816 on or separate from display 810. General purpose I/O interface 812 also connects to a variety of peripherals 818 including printers and scanners, such as an OfficeJet or DeskJet from Hewlett Packard.

A sound controller 820 is also provided in the computing device, such as Sound Blaster X-Fi Titanium from Creative, to interface with speakers/microphone 822 thereby providing sounds and/or music. The general purpose storage controller 824 connects the storage medium disk 804 with communication bus 826, which may he an ISA, EISA, VESA, PCI, or similar, for interconnecting all of the components of the computing device. A description of the general features and functionality of the display 810, keyboard and/or mouse 814, as well as the display controller 808, storage controller 824, network controller 806, sound controller 820, and general purpose I/O interface 812 is omitted herein for brevity as these features are known.

The computing devices used with embodiments described herein may not include all features described in FIG. 8. In addition, other features used with embodiments described herein may not be described with reference to FIG. 8.

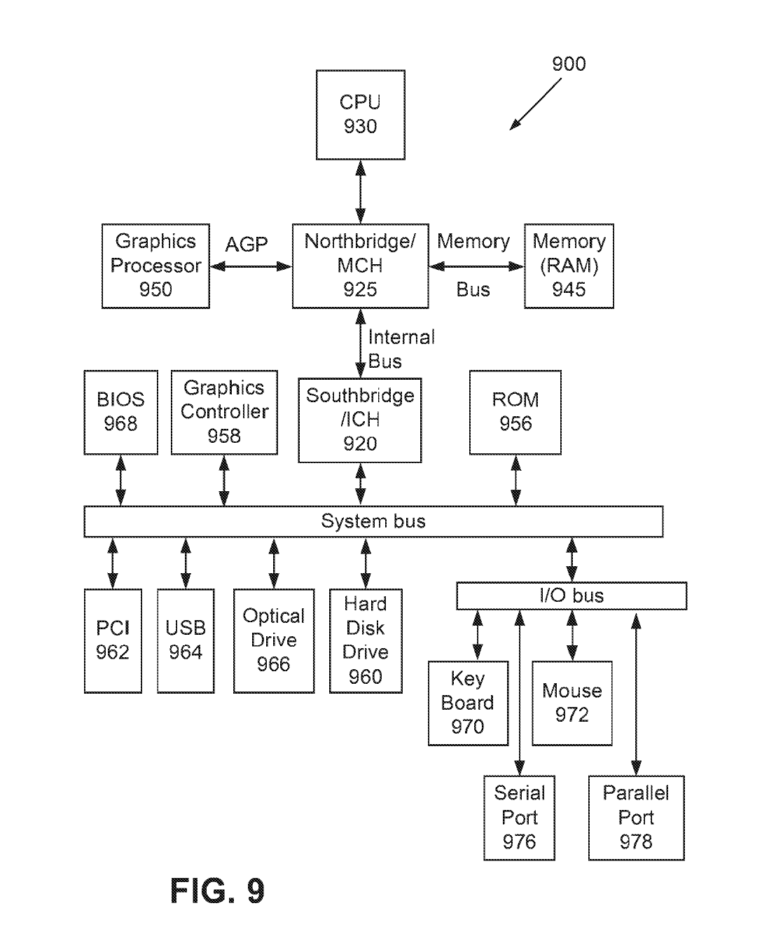

FIG. 9 is a schematic diagram of an exemplary data processing system, according to certain embodiments described herein. The data processing system is an example of a computer in which code or instructions implementing the processes of the illustrative embodiments can be executed.

In FIG. 9, data processing system 900 employs an application architecture including a north bridge and memory controller application (NB/MCH) 925 and a south bridge and input/output (I/O) controller application (SB/ICH) 920. The central processing unit (CPU) 930 is connected to NB/MCH 925. The NB/MCH 925 also connects to the memory 945 via a memory bus, and connects to the graphics processor 950 via an accelerated graphics port (AGP). The NB/MCH 925 also connects to the SB/ICH 920 via an internal bus (e.g., a unified media interface or a direct media interface). The CPU 930 can include one or more processors and/or can be implemented using one or more heterogeneous processor systems.

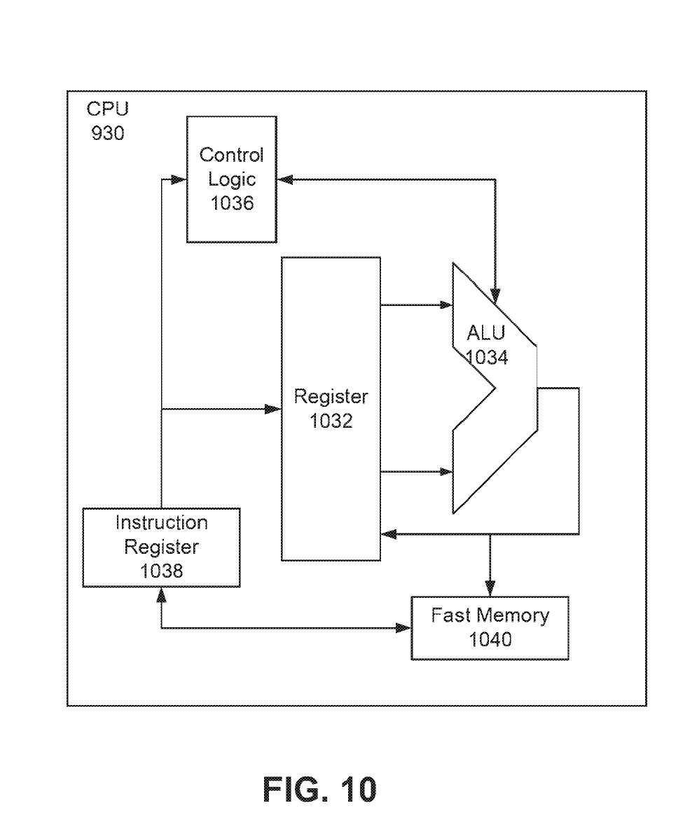

For example, FIG. 10 shows one implementation of CPU 930. In one implementation, an instruction register 1038 retrieves instructions from a fast memory 1040. At least part of these instructions are fetched from an instruction register 1038 by a control logic 1036 and interpreted according to the instruction set architecture of the CPU 930, Part of the instructions can also be directed to a register 1032. In one implementation the instructions are decoded according to a hardwired method, and in another implementation the instructions are decoded according to a microprogram that translates instructions into sets of CPU configuration signals that are applied sequentially over multiple clock pulses.

After fetching and decoding the instructions, the instructions are executed using an arithmetic logic unit (ALU) 1034 that loads values from the register 1032 and performs logical and mathematical operations on the loaded values according to the instructions. The results from these operations can be fed back into the register 1032 and/or stored in a fast memory 1040. According to certain implementations, the instruction set architecture of the CPU 930 can use a reduced instruction set architecture, a complex instruction set architecture, a vector processor architecture, or a very large instruction word architecture. Furthermore, the CPU 930 can be based on the Von Neuman model or the Harvard model. The CPU 930 can be a digital signal processor, an FPGA, an ASIC, a PLA, a PLD, or a CPLD. Further, the CPU 930 can be an x86 processor by Intel or by AMD; an ARM processor; a Power architecture processor by, e.g., IBM; a SPARC architecture processor by Sun Microsystems or by Oracle; or other known CPU architectures.

Referring again to FIG. 9, the data processing system 900 can include the SB/ICH 920 being coupled through a system bus to an I/O bus, a read only memory (ROM) 956, universal serial bus (USB) port 964, a flash binary input/output system (BIOS) 968, and a graphics controller 958. PCI/PCIe devices can also be coupled to SB/ICH 920 through a PCI bus 962.

The PCI devices can include, for example, Ethernet adapters, add-in cards, and PC cards for notebook computers. The Hard disk drive 960 and CD-ROM 966 can use, for example, an integrated drive electronics (IDE) or serial advanced technology attachment (SATA) interface. In one implementation the I/O bus can include a super I/O (SIO) device.

Further, the hard disk drive (HDD) 960 and optical drive 966 can also be coupled to the SB/ICH 920 through a system bus. In one implementation, a keyboard 970, a mouse 972, a parallel port 978, and a serial port 976 can be connected to the system bus through the I/O bus. Other peripherals and devices can be connected to the SB/ICH 920 using a mass storage controller such as SATA or PATA, an Ethernet port, an ISA bus, a LPC bridge, SMBus, a DMA controller, and an. Audio Codec. In an embodiment, peripheral devices can be connected to processor 350 for downloading of stored data retrieved by the sleep-monitoring cap.

Moreover, the present disclosure is not limited to the specific circuit elements described herein, nor is the present disclosure limited to the specific sizing and classification of these elements. For example, the skilled artisan will appreciate that the circuitry described herein may be adapted based on changes on battery sizing and chemistry, or based on the requirements of the intended back-up load to be powered.

The functions and features described herein may also be executed by various distributed components of a system. For example, one or more processors may execute these system functions, wherein the processors are distributed across multiple components communicating in a network. For example, distributed performance of the processing functions can be realized using grid computing or cloud computing. Many modalities of remote and distributed computing can be referred to under the umbrella of cloud computing, including: software as a service, platform as a service, data as a service, and infrastructure as a service. Cloud computing generally refers to processing performed at centralized locations and accessible to multiple users who interact with the centralized processing locations through individual terminals.

Many advantages are provided by the sleep-monitoring cap having circuitry configured to monitor and detect a sleeping disorder, such as narcolepsy. An accurate detection of narcolepsy usually requires one or more days and nights at a sleep disorder facility, which can be expensive and time consuming. Embodiments described herein provide a portable inexpensive alternative outside of the sleep disorder facility. Sleep activity can be monitored in the home of the person, instead of a medical facility. Data retrieved by the processor 350 can be examined by medical personnel to determine a possible sleep disorder of the person using the sleep-monitoring cap. In particular, embodiments described herein can monitor and detect narcolepsy of the person using the sleep-monitoring cap.

Many advantages are also provided by the sleep-monitoring cap having circuitry configured to monitor and detect drowsiness of a user wearing the sleep-monitoring cap. Drowsiness affects many people in many different environments, such as while driving or operating equipment, while working at a task, and while listening to a speaker. Embodiments described herein can help keep the person wearing the sleep-monitoring cap away from danger, keep the person alert to perform a given task, and save the person embarrassment from falling asleep at a meeting or lecture, respectively.

Embodiments described herein for a sleep-monitoring cap transmit signals to a light-emitting device when brain wave activity, indicative of drowsiness is detected. This provides an indicator to others near to the person wearing the sleep-monitoring cap of his/her drowsiness. Embodiments also provide a sleep-monitoring cap to detect drowsiness in which the feedback is provided only to the person wearing the sleep-monitoring cap. The sleep-monitoring cap can be thin, so as to fit underneath a regular hat. This would conceal most or all of the sleep-monitoring cap from view by others.

The foregoing discussion discloses and describes merely exemplary embodiments of the present disclosure. As will be understood by those skilled in the art, the present disclosure may be embodied in other specific forms without departing from the spirit or essential characteristics thereof. Accordingly, the present disclosure is intended to be illustrative and not limiting thereof. The disclosure, including any readily discernible variants of the teachings herein, defines in part, the scope of the foregoing claim terminology.

1. A sleep-monitoring cap, comprising:
a plurality of interconnected electrodes embedded within a body of the sleep-monitoring cap, wherein the plurality of interconnected electrodes are located at positions across a central transverse region, below and along a side of each eye, and on a rear mid-region of a person's head when the person is wearing the sleep-monitoring cap;
a vibratory device embedded within the body of the sleep-monitoring cap, wherein the vibratory device is connected to the plurality of interconnected electrodes;
first processing circuitry embedded within the body of the sleep-monitoring cap, wherein the first processing circuitry is configured to
monitor, convert, process, and store a first set of brain wave activity retrieved by the plurality of interconnected electrodes from the person wearing the sleep-monitoring cap,
determine whether a sleep state exists from the monitored first set of brain wave activity,
when the sleep state exists, determine whether a first sleep stage is a rapid eye movement (REM) sleep stage,
when the first sleep stage is a REM sleep stage, record the sleep state as a sleep onset REM period, and
activate the vibratory device after a pre-determined time period when the sleep onset REM period has been recorded; and
second processing circuitry embedded within the body of the sleep-monitoring cap, wherein the second processing circuitry is configured to
monitor, convert, process, and store a second set of brain wave activity retrieved by the plurality of interconnected electrodes from the person wearing the sleep-monitoring cap,
determine whether the monitored second set of brain wave activity includes low amplitude mixed-frequency waves;
when the monitored second set of brain wave activity includes the low amplitude mixed-frequency waves, activate the vibratory device;
determine whether the monitored second set of brain wave activity includes theta waves followed by vertex sharp waves; and
when the monitored second set of brain wave activity includes the theta waves followed by the vertex sharp waves, activate the vibratory device.

2. The sleep-monitoring cap of claim 1, wherein the first processing circuitry is further configured to monitor and determine a narcoleptic state of the person wearing the sleep-monitoring cap.

3. The sleep-monitoring cap of claim 1, wherein the second processing circuitry is further configured to monitor and determine a drowsy state of the person wearing the sleep-monitoring cap.

4. The sleep-monitoring cap of claim 1, wherein the second processing circuitry is further configured to transmit a wireless signal to a light-emitting device when the vibratory device is activated.

5. The sleep-monitoring cap of claim 4, wherein the second processing circuitry is further configured to transmit the wireless signal to the light-emitting device located on a moving vehicle operated by the person wearing the sleep-monitoring cap.

6. A sleep-monitoring cap, comprising:
a plurality of interconnected electrodes embedded within a body of the sleep-monitoring cap, wherein the plurality of interconnected electrodes are located at positions across a central transverse region, below and along a side of each eye, and on a rear mid-region of a person's head when the person is wearing the sleep-monitoring cap;
a vibratory device embedded within the body of the sleep-monitoring cap, wherein the vibratory device is connected to the plurality of interconnected electrodes; and
processing circuitry embedded within the body of the sleep-monitoring cap, wherein the processing circuitry is configured to
monitor, convert, process, and store brain wave activity retrieved by the plurality of interconnected electrodes from the person wearing the sleep-monitoring cap,
determine whether a sleep state exists from the monitored brain wave activity,
when the sleep state exists, determine whether a first sleep stage is a rapid eye movement (REM) sleep stage,
when the first sleep stage is a REM sleep stage, record the sleep state as a sleep onset REM period, and
activate the vibratory device after a pre-determined time period when the sleep onset REM period has been recorded.

7. The sleep-monitoring cap of claim 6, further comprising:
a transmitter embedded within the body of the sleep-monitoring cap, wherein the transmitter is configured to transmit a signal to a light-emitting device when the vibratory device is activated.

8. The sleep-monitoring cap of claim 7, wherein the transmitter is configured to transmit a wireless signal to the light-emitting device.

9. The sleep-monitoring cap of claim 8, wherein the processing circuitry further configured to activate a communication to one or more communication devices when the vibratory device is activated.

10. The sleep-monitoring cap of claim 6, wherein the vibratory device is located at a rear base portion of the sleep-monitoring cap.

11. The sleep-monitoring cap of claim 6, wherein the processing circuitry is configured to activate the vibratory device when a narcoleptic state is determined.

12. The sleep-monitoring cap of claim 6, further comprising:
a battery-operated power source.

13. A sleep-monitoring cap comprising:
a plurality of interconnected electrodes embedded within a body of the sleep-monitoring cap, wherein the plurality of interconnected electrodes are located at positions across a central transverse region, below and along a side of each eye, and on a rear mid-region of a person's head when the person is wearing the sleep-monitoring cap;
a vibratory device embedded within the body of the sleep-monitoring cap, wherein the vibratory device is connected to the plurality of interconnected electrodes; and
processing circuitry embedded within the body of the sleep-monitoring cap, wherein the processing circuitry is configured to
monitor, convert, process, and store brain wave activity retrieved by the plurality of interconnected electrodes from the person wearing the sleep-monitoring cap,
determine whether the monitored brain wave activity includes low amplitude mixed-frequency waves;
when the monitored brain wave activity includes the low amplitude mixed-frequency waves, activate the vibratory device;
determine whether the monitored brain wave activity includes theta waves followed by vertex sharp waves; and
when the monitored brain wave activity includes the theta waves followed by the vertex sharp waves, activate the vibratory device.

14. The sleep-monitoring cap of claim 13, wherein the processing circuitry is further configured to continue to activate the vibratory device until an awakened state of the person is monitored.

15. The sleep-monitoring cap of claim 13, wherein the processing circuitry is further configured to change one of an intensity of vibration or a pattern of vibration until an awakened state of the person is monitored.

16. The sleep-monitoring cap of claim 13, wherein the sleep-monitoring cap is configured to monitor and determine a drowsy state of a person wearing the sleep-monitoring cap.

17. The sleep-monitoring cap of claim 13, wherein the processing circuitry is further configured to transmit a signal to a light-emitting device when the vibratory device is activated.

18. The sleep-monitoring cap of claim 17, wherein the processing circuitry is further configured to transmit the signal wirelessly to the light-emitting device located on the sleep-monitoring cap.

19. The sleep-monitoring cap of claim 17, wherein the processing circuitry is further configured to transmit the signal wirelessly to the light-emitting device located on a moving vehicle operated by the person wearing the sleep-monitoring cap.

20. The sleep-monitoring cap of claim 13, wherein the processing circuitry is further configured to transmit a communication to one or more communication devices when the vibratory device is activated.
